(19) United States
(12) Patent Application Publication
(10) Pub. No.: US 2016/0166802 A1
(43) Pub. Date: Jun. 16, 2016

(54) ANTIBACTERIAL CATHETER

(71) Applicant: BIO-MEDICAL CARBON TECHNOLOGY CO., LTD., Taichung City (TW)

(72) Inventors: Tse-Hao KO, TAIPEI CITY (TW); Ming-Chean HUNG, TAICHUNG CITY (TW); Jui-Hsiang LIN, TAICHUNG CITY (TW); Yen-Ju SU, TAICHUNG CITY (TW)

(21) Appl. No.: 14/569,064

(22) Filed: Dec. 12, 2014

(51) Int. Cl. A61M 25/00; A61L 29/08; A61L 29/16; A61L 29/10

(52) U.S. Cl. CPC A61M 25/0017; A61L 29/10; A61L 29/085; A61L 29/16

(57) ABSTRACT

An antibacterial catheter includes a body having a urine passage, and an antibacterial carbon material disposed in the urine passage. The antibacterial carbon material has a BET specific surface area in a range between 500 m²/g and 1800 m²/g, and contains a plurality of pores including micropores that have a diameter less than 2 nm. The antibacterial carbon material has a micropore-to-pore volume ratio ranging from 30% to 50%. Because the antibacterial carbon material is disposed in the urine passage of the catheter, the bacteria outside the human body may not easily enter into the urinary bladder and the bacteria inside the urinary bladder can be destroyed, such that the urinary system can be effectively prevented from the bacterial infection.

### BACKGROUND OF THE INVENTION

1. Field of the Invention

The present invention relates generally to catheters and more particularly, to an antibacterial catheter for effectively preventing the bacterial infection of the urinary system.

2. Description of the Related Art

A conventional catheter includes a rubber tube and a balloon disposed thereon. By means of water-inflated balloon, the catheter can be fixed in position in the urinary bladder. Such urethral catheter that is allowed to remain in the urinary bladder for a long time is called an indwelling catheter.

According to a statistic, 25% of patients in hospital may use the indwelling catheter, among which there are about 3-10% of patients who have urinary infection every day. In addition, the patient may have septicemia as the urinary infection is serious. On the other hand, a statistic of Taiwan shows that the ratio of the patient with urinary infection reaches up to 36.5% on average during 10 years from 2003 to the third-quarter of 2012, which is the first ranking in all kinds of infections in medical centers. Therefore, it is apparent that the usage of the conventional indwelling catheter may increase the risk of exogenous bacterial infection.

### SUMMARY OF THE INVENTION

It is an objective of the present invention to provide an antibacterial catheter, which is capable of effectively inhibiting the growth of bacteria thereby preventing the bacterial infection of the urinary system.

To attain the above objective, the present invention provides an antibacterial catheter which comprises a body and an antibacterial carbon material. The body has a urine passage, an inlet located at an end of the urine passage, and an outlet located at the other end of the urine passage. The urine passage communicates with the inlet and the outlet. The antibacterial carbon material is disposed in the urine passage, has a BET (Brunauer-Emmett-Teller) specific surface area in a range between 500 m²/g and 1800 m²/g, and comprises a plurality of pores including micropores having a diameter less than 2 nm. The antibacterial carbon material has a micropore-to-pore volume ratio, i.e. the ratio of total volume of the micropores to total volume of the pores, ranging from 30% to 50%.

Because the antibacterial carbon material is disposed inside the urine passage of the antibacterial catheter, the bacteria outside the human body may not easily enter into the urinary bladder through the urine passage and the bacteria inside the urinary bladder can be eliminated, such that the urinary system can be effectively prevented from the bacterial infection. Therefore, the bacterial infection of the urinary bladder caused by the conventional catheter can be improved.

### BRIEF DESCRIPTION OF THE DRAWINGS

FIG. 1 is a cross-sectional view of an antibacterial catheter according to a first preferred embodiment of the present invention.

FIG. 2 is a scanning electron microscopic (SEM) image of an antibacterial carbon material of test 1, showing the initial attachment of Escherichia coli (E. coli) to the antibacterial carbon material of test 1.

FIG. 3 is an SEM image of the antibacterial carbon material of test 1, showing the attachment of E. coli to the antibacterial carbon material of test 1 for 24 hours.

FIG. 4 is an SEM image of an antibacterial carbon material of test 2, showing the initial attachment of E. coli to the antibacterial carbon material of test 2.

FIG. 5 is an SEM image of the antibacterial carbon material of test 2, showing the attachment of E. coli to the antibacterial carbon material of test 2 for 24 hours.

FIG. 6 is a cross-sectional view of an antibacterial catheter according to a second preferred embodiment of the present invention.

FIG. 7 is a cross-sectional view of an antibacterial catheter according to a third preferred embodiment of the present invention.

### DETAILED DESCRIPTION OF EMBODIMENTS

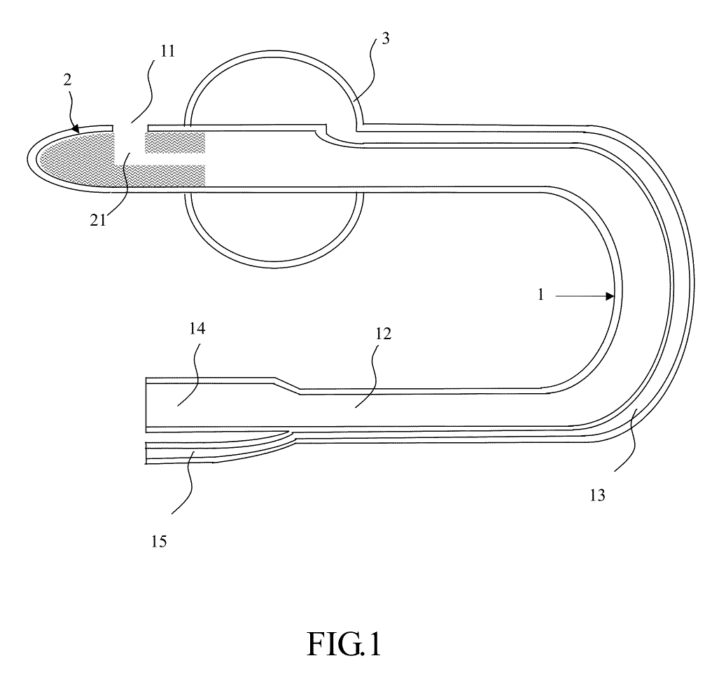

The structure and effect of the present invention will become more fully understood from the detailed description given herein below and the accompanying drawings. Referring to FIG. 1, an antibacterial catheter according to a first preferred embodiment of the present invention comprises a body 1 and an antibacterial carbon material 2 disposed in the body 1. The antibacterial catheter is applicable to be placed in a patient's urinary bladder. The front portion of the body 1 accommodating the antibacterial carbon material 2 is adapted to be placed inside the patient's urinary bladder in a way that the middle and rear portions of the body 1 are located outside the patient's body.

The body 1 is formed of a flexible material, such as silica gel or polyethylene chloride, and comprises an inlet 11, a urine passage 12 formed inside the body 1, an outlet 14, a balloon 3 disposed outside the urine passage 12 and adjacent to the inlet 11, a drainage channel 13 disposed beside the urine passage 12, and an injection port 15. The inlet 11 is located at the front end of the urine passage 12 for introducing the urine from the urinary bladder into the urine passage 12. The outlet 14 is located at the rear end of the urine passage 12 and exposed to an outside of the human body for draining the urine from the urine passage 12 into a urine collection bag. The urine passage 12 is communicated with the inlet 11 and the outlet 14 respectively. The urine passage 12 and the drainage channel 13 are not communicated to each other. The injection port 15, the drainage channel 13 and the balloon 3 are communicated together. The injection port 15 is adapted to be connected to a syringe, such that the medical staff can inject physiological saline into the balloon 3 to expand the same, thereby fixing the balloon 3 and the front portion of the body 1 in the patient's urinary bladder and thus the antibacterial catheter may not be easily separated therefrom.

The antibacterial carbon material 2 is disposed at the front end of the urine passage 12 and adjacent to the inlet 11. In addition, the antibacterial carbon material 2 has an antibacterial passage 21 communicated with the inlet 11 and the urine passage 12. As such, because the urine may first flow through the antibacterial passage 21 when it flows into the body 1 from the inlet 11, and then flow out of the urine passage 12 from the outlet 14, any bacteria present in the urine or the urine passage 12 can be eliminated through the attachment of the bacteria to the antibacterial carbon material 2. It's worth noting that the antibacterial carbon material 2 may be polyacrylonitrile (PAN) activated carbon, bamboo charcoal, or graphene, and may be in woven, non-woven, or powder form. As the antibacterial carbon material is in powder form, each particle may have a length of 10 μm to 300 μm and a diameter of 5 μm to 15 μm. The antibacterial carbon material 2 has a BET specific surface area of between 500 m²/g and 1800 m²/g, and contains a plurality of pores including micropores. It is to be noted that the micropore defined throughout the specification and appendix claims indicates the pore having a diameter less than 2 nm. Further, the antibacterial carbon material 2 has a micropore-to-pore volume ratio, which is defined as the ratio of total volume of the micropores to total volume of the pores throughout the specification and appendix claims, ranging from 30% to 50%.

In order to understand the antibacterial property of the antibacterial carbon material 2, the antibacterial carbon material 2 is tested and observed by the following apparatus and method.

Specifically, a Cold Field Emission Scanning Electron Microscope and Energy Dispersive Spectrometer (S-4800, supplied by HITACHI, LTD. Japan) is used to observe the surface of the carbon material sample. A suitable sized carbon material sample is fixed on the stage having a diameter of 2.5 cm by a carbon tape, and then the carbon material sample is baked on a heating plate at 80° C. for one hour. After that, the surface of the carbon material sample is observed by the aforesaid spectrometer at an accelerating voltage of 10 kV to 15 kV at a magnification of 5,000 times.

The BET (Brunauer-Emmett-Teller) specific area of the carbon material sample is determined by a surface area analyzer (Micromeritics ASAP 2020, supplied by Micromeritics Instrument Corporation, America). The carbon material sample is vacuumized at a high temperature (360° C.), and then the adsorption gas (nitrogen) is filled. The experimental temperature is maintained at 77K and the experimental pressure is maintained at 760 mm-Hg.

As regards the antibacterial analysis, six circular carbon material samples with a diameter of 4.8±0.1 cm are sterilized at 121° C. and 103 kPa (1.05 kg/m²) for 20 minutes, and then are tested according to AATCC 100-1998 antibacterial standard method.

In test 1, an oxidized PAN fiber cloth was activated under a nitrogen atmosphere. The process temperature was increased from room temperature to 1,000° C. at a rate of 4° C. per minute. After the water vapor activation was carried out for 10 minutes, the process temperature was decreased to room temperature at a rate of 10° C. per minute. A PAN activated carbon fiber cloth, which has a BET specific surface area of 1050 m²/g and a micropore-to-pore volume ratio of 45%, is finally obtained. The PAN activated carbon fiber cloth of test 1 was used to make the antibacterial carbon material 2 and then the antibacterial analysis was performed.

In test 2, a PAN activated carbon fiber cloth containing silver nanoparticles is manufactured. The activated carbon fiber cloth of test 1 was immersed in 0.01M silver nitrate solution with stirring for 0.5 hour at 50 rpm. Then, the dehydration process was conducted at 120° C. to remove water. Thereafter, the process temperature was increased from room temperature to 600° C. at a rate of 4° C. per minute under a nitrogen atmosphere to conduct thermal decomposition process for 1 minute. After thermal decomposition, the process temperature was decreased to room temperature at a rate of 10° C. per minute. The product thus obtained was washed with water for 2 hours followed by conducting the dehydration process at 120° C. for 2 hours. Finally, the activated carbon fiber cloth containing 0.06 wt % silver nanoparticles that have a diameter of between 10 nm and 50 nm was produced. The activated carbon fiber cloth containing silver nanoparticles has a true density of 2.06 g/cm³, a carbon content of 85.5 wt %, an oxygen content of 10.4 wt %, a BET specific surface area of 1030 m²/g, and a micropore-to-pore volume ratio of 44%. The activated carbon fiber cloth containing silver nanoparticles of test 2 was used to make the antibacterial carbon material 2 and then the antibacterial analysis was performed. The results of the antibacterial analysis of test 1 and test 2 were shown in Table 1.

TABLE 1
Results of antibacterial analysis

| Antibacterial | | |
|---|---|---|
| Carbon | | Antibacterial |
| Material | Tested Strains | Rate (%) |
| Test 1 | Pseudomonas aeruginosa | 99.0 |
| | Staphylococcus aureus | 99.3 |
| Test 2 | pneumobacillus | >99.99 |
| | Streptococcus pneumoniae | >99.99 |
| | E. coli | >99.99 |
| | Pseudomonas aeruginosa | >99.99 |
| | methicillin resistant Staphylococcus Aureu | >99.99 |
| | multidrug-resistant Psuedomonas aeruginosa | >99.99 |

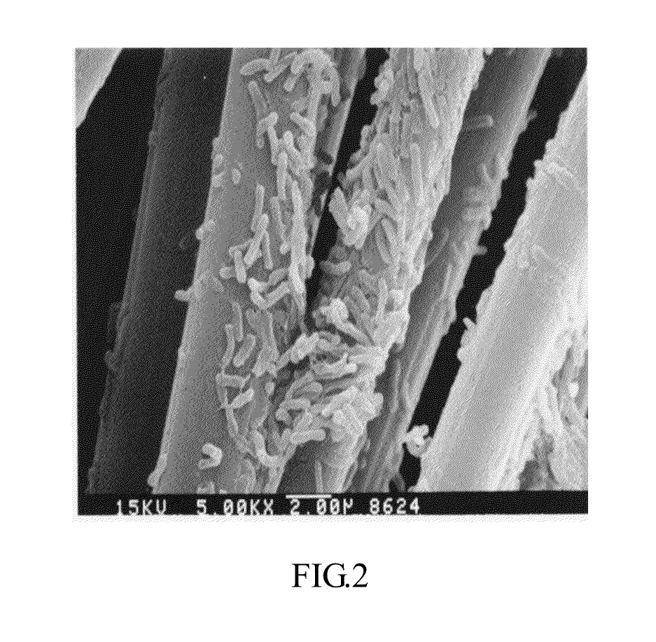

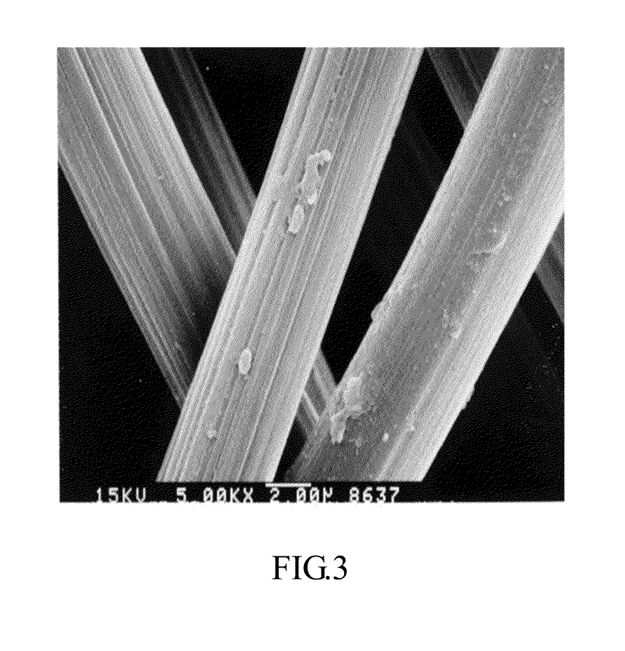

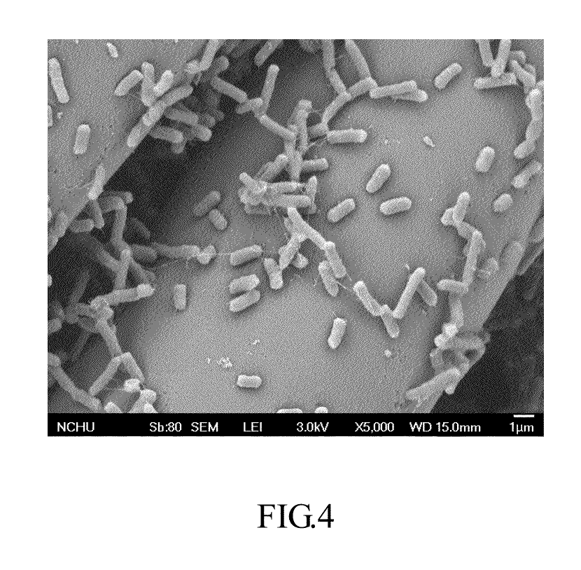

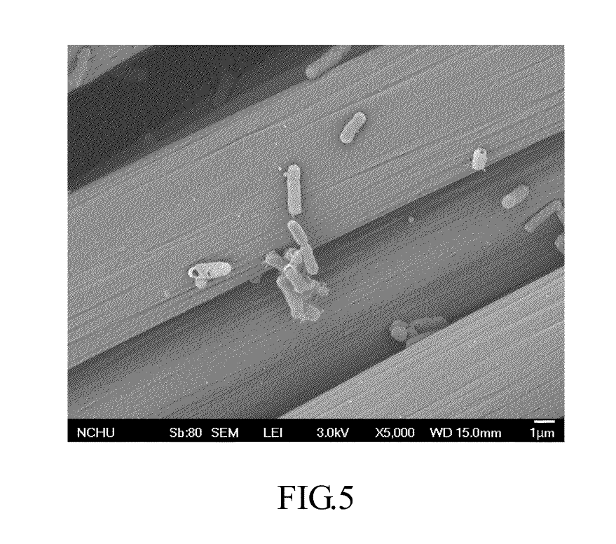

The antibacterial activities of test 1 and test 2 shown in Table 1 were evaluated according to AATCC 100 test method. In Table 1, it is apparent that the antibacterial rate of test 1 against Staphylococcus aureus (S. aureus) is 99.3%, and against Pseudomonas aeruginosa (P. aeruginosa) is 99.0%. In addition, FIG. 2 is the SEM image of the antibacterial carbon material of test 1, showing the initial attachment of Escherichia coli thereto, and FIG. 3 is the SEM image of the antibacterial carbon material of test 1, showing the attachment of Escherichia coli thereto for 24 hours. In comparison with FIG. 2 and FIG. 3, the amount of Escherichia coli shown in FIG. 3 is significantly reduced. On the other hand, the antibacterial activity of the activated carbon fiber cloth containing 0.06 wt % silver nanoparticles of test 2 can be enhanced to more than 99.99%. FIG. 4 is the SEM image of the antibacterial carbon material of test 2, showing the initial attachment of Escherichia coli thereto, and FIG. 5 is the SEM image of the antibacterial carbon material of test 2, showing the attachment of Escherichia coli thereto for 24 hours. As can be seen in FIGS. 4-5, because most of the Escherichia coli originally attached to the antibacterial carbon material 2 have been destroyed, it is confirmed that silver is helpful in killing bacteria on PAN activated carbon fiber cloth, and thus the antibacterial rate can be enhanced.

For the purpose of understanding the influence on the antibacterial activity from the specific surface area of the antibacterial carbon material, the oxidized PAN fiber clothes were activated, under the same condition as set forth in test 1 but the activation temperature were 600° C., 800° C. and 1200° C. respectively, to manufacture the PAN activated carbon fiber clothes. The carbon fiber clothes thus obtained are respectively used as the antibacterial carbon material and designated as control group, test 3 and test 4. After evaluation, the BET specific surface areas are 380 m²/g, 650 m²/g and 1800 m²/g respectively, and the micropore-to-pore volume ratios are 27%, 41% and 48% for control group, test 3 and test 4 respectively.

The antibacterial activities of the control group, test 3 and test 4 shown in Table 2 were evaluated according to AATCC 100 test method. As shown in Table 2 and the aforesaid evaluation result, the control group, which has the lowest BET specific surface area and micropore-to-pore volume ratio, has an antibacterial activity obviously lower than that of test 3 or test 4 that has a higher BET specific surface area.

TABLE 2
Results of antibacterial analysis

| | | | | Antibacterial |
|---|---|---|---|---|
| | Activation | BET Specific | Antibacterial | Rate of |
| | Temperature | Surface Area | Rate of | P. Aeruginosa |
| | (° C.) | (m²/g) | S. Aureus (%) | (%) |
| Control | 600 | 380 | 91.8 | 91.2 |
| group | | | | |
| Test 3 | 800 | 650 | 99.9 | 99.9 |
| Test 4 | 1200 | 1800 | >99.9 | >99.9 |

From the above mentioned experimental results, the antibacterial carbon material may have better antibacterial activity when the antibacterial carbon material has the BET specific surface area of between 500 m²/g and 1800 m²/g and the micropore-to-pore volume ratio ranging from 30% to 50%. Further, the antibacterial carbon material having the BET specific surface area of between 650 m²/g and 1800 m²/g may have superior antibacterial activity.

Organic or inorganic antibacterial treatment can be employed to improve the antibacterial activity of the antibacterial carbon material 2. Specifically, as regards the organic antibacterial treatment, the antibacterial carbon material 2 may be immersed in or coated with an aminoglycoside substance such as gentamicin, tobramycin, amikacin, etc., such that the antibacterial carbon material 2 may be combined with the organic substance to form the material having high antibacterial activity. The urinary system can be effectively prevented from the bacterial infection through applying the antibacterial carbon material 2 thus treated in the catheter of the present invention. The organic antibacterial treatment is preferably conducted by employing gentamicin to obtain a better antibacterial activity.

For inorganic antibacterial treatment, the inorganic antibacterial metal such as gold, silver, copper or zinc may be combined with the antibacterial material 2 through immersing, coating, electroplating, evaporation, chemical vapor deposition, electroless plating, etc. to form the material having high antibacterial activity. The antibacterial carbon material 2 thus treated contains 0.01-0.1 wt % gold, silver, copper or zinc. As such, the urinary system can be effectively prevented from the bacterial infection through applying the antibacterial carbon material 2 thus treated in the catheter of the present invention. The inorganic antibacterial treatment is preferably conducted by employing silver or copper to obtain a better antibacterial activity.

Regarding the embodiment of the structure of the antibacterial carbon material 2 of the present invention, it may be formed by rolling an activated carbon fiber cloth into a cylindrical shape, or grinding an activated carbon fiber material into a powder form and then attaching the powders thus obtained on an inner wall of the body 1. In addition, a PU-foamed material containing the activated carbon fibers in a rod-like shape may also be used as the antibacterial carbon material 2.

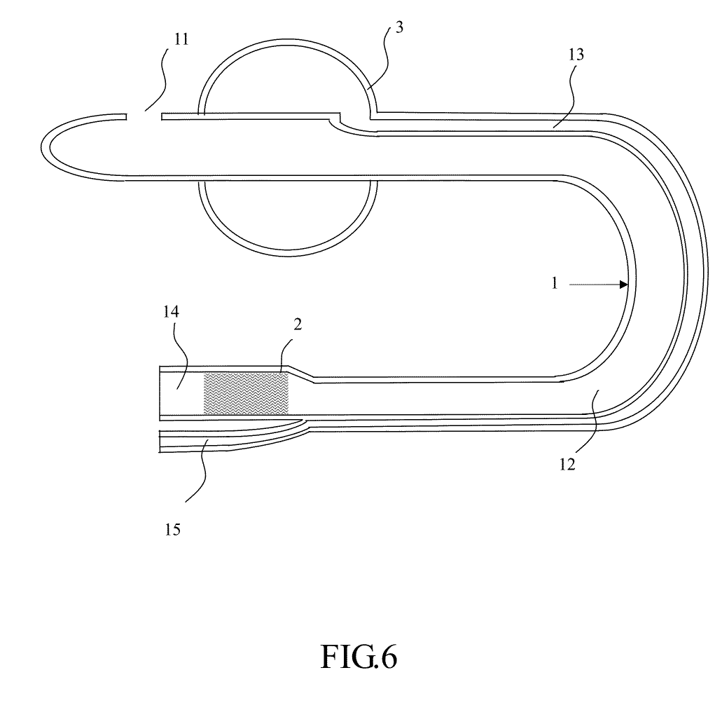

The antibacterial catheter of the second preferred embodiment of the present invention is shown in FIG. 6 and is substantially the same as that of the first preferred embodiment except that the antibacterial carbon material 2 is disposed adjacent to the outlet 14. The urine introduced into the antibacterial passage 21 may flow through the antibacterial carbon material 2 and finally flow out from the outlet 14. As such, any bacteria present in the urine or the urine passage 12 may be attached to the antibacterial carbon material 2 and be destroyed after a period of time due to the antibacterial activity of the antibacterial carbon material 2.

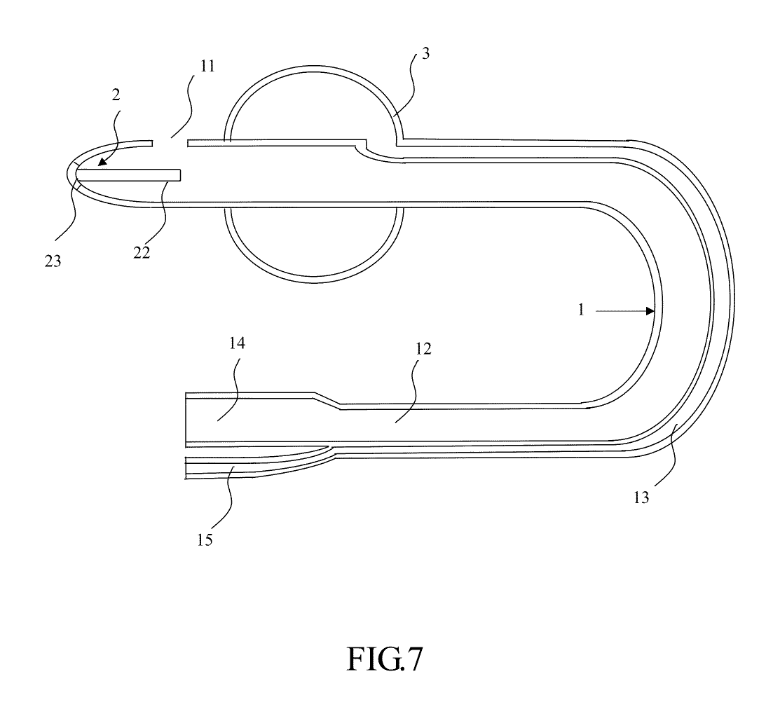

The antibacterial catheter of the third preferred embodiment of the present invention is shown in FIG. 7 and is substantially the same as that of the first preferred embodiment except that the antibacterial carbon material 2 comprises a fixing portion 23 mounted to the front end of the urine passage 12 and adjacent to the inlet 11, and an extending portion 22 extending from the fixing portion 23 along the urine passage 12. In this embodiment, the extending portion 22 is cylindrical or rod-like. As such, any bacteria present in the urine or the urine passage 12 may be attached to the extending portion 22 of the antibacterial carbon material 2 and be destroyed after a period of time due to the antibacterial activity of the antibacterial carbon material 2.

In conclusion, the antibacterial catheter of the present invention possesses the following advantages: the bacteria outside the human body may not easily enter into the urinary bladder through the urine or the urine passage since the antibacterial carbon material 2 disposed in the urine passage 12 plays a role to interrupt the conveyance of the bacteria, thereby effectively preventing the bacterial infection of the urinary system and bacterial cystitis caused by the conventional indwelling catheter.

What is claimed is:

1. An antibacterial catheter comprising:
a body including a urine passage, an inlet located at an end of the urine passage, and an outlet located at the other end of the urine passage, the urine passage being in fluid communication with the inlet and the outlet; and
an antibacterial carbon material disposed in the urine passage and having a BET specific surface area ranging from 500 m²/g to 1800 m²/g, the antibacterial carbon material comprising a plurality of pores including micropores that have a diameter less than 2 nm, the antibacterial carbon material having a micropore-to-pore volume ratio ranging from 30% to 50%, wherein the micropore-to-pore volume ratio is defined as the ratio of a total volume of the micropores to a total volume of the pores.

2. The antibacterial catheter as claimed in claim 1, wherein the antibacterial carbon material is made of polyacrylonitrile activated carbon, bamboo charcoal, or graphene.

3. The antibacterial catheter as claimed in claim 2, wherein the antibacterial carbon material is in woven, non-woven, or powder form.

4. The antibacterial catheter as claimed in claim 1, wherein the antibacterial carbon material has the BET specific surface area ranging from 650 m²/g to 1800 m²/g.

5. The antibacterial catheter as claimed in claim 1, wherein the body further comprises a balloon disposed outside the urine passage and adjacent to the inlet, a drainage channel disposed beside the urine passage and not in communication with the urine passage, and an injection port; wherein the balloon, the drainage channel and the injection port are in fluid communication together.

6. The antibacterial catheter as claimed in claim 1, wherein the antibacterial carbon material is disposed adjacent to the inlet.

7. The antibacterial catheter as claimed in claim 6, wherein the antibacterial carbon material comprises an antibacterial passage in fluid communication with the inlet and the urine passage.

8. The antibacterial catheter as claimed in claim 1, wherein the antibacterial carbon material is disposed adjacent to the outlet.

9. The antibacterial catheter as claimed in claim 1, wherein the antibacterial carbon material comprises a fixing portion mounted to the body and adjacent to the inlet, and an extending portion extending from the fixing portion along the urine passage.

10. The antibacterial catheter as claimed in claim 1, wherein the antibacterial carbon material contains 0.01-0.1 wt % gold, silver, copper or zinc.
